(19) United States
(12) Patent Application Publication
(10) Pub. No.: US 2014/0274727 A1
(43) Pub. Date: Sep. 18, 2014

(54) CARIES GENETIC PREDISPOSITION ANALYSIS BY MOLECULAR TESTING

(71) Applicant: Daniel R. Cohen, Ramsey, NJ (US)

(72) Inventor: Daniel R. Cohen, Ramsey, NJ (US)

(73) Assignee: Dental Gene ID, LLC, Ramsey, NJ (US)

(21) Appl. No.: 13/799,747

(22) Filed: Mar. 13, 2013

(51) Int. Cl. C12Q 1/68

(52) U.S. Cl. CPC C12Q 1/6883
USPC 506/2

(57) ABSTRACT

Means and methods of predicting future dental problems are disclosed. Molecular tests use biological matter from a patient to obtain information regarding various genes. The tests and methods generate displays and reports regarding genetic risk pertaining to cavities, tooth decay and other disorders.

### BACKGROUND OF THE INVENTION

(1) Field of the Invention

The invention generally relates to molecular testing. More particularly, the invention relates to means and methods of sequencing single nucleotides polymorphisms assigned to a high risk for caries development or tooth decay.

(2) Description of the Related Art

U.S. Patent Publication 2009/0299645 by Colby et al discloses general methods of generating genetic profiles and health scores. U.S. Pat. No. 7,955,808 by Bufe et al relates to testing subjects for genes related to bitter-taste receptors. U.S. Pat. No. 8,017,334 by Takao et al discloses a method for testing individuals for dysgeusia by review of taste receptor genes. While the known prior art does review various aspects of genes TAS2R38 and TAS1R2, the following SNPs: rs635808, rs17057381, rs4251631, rs118500320, rs9793739, rs1383934, rs11099896, and any other SNP and/or gene linked to caries predisposition, there are no genetic tests in the prior art for testing individuals for a predisposition to caries or tooth decay, thus, there is a need in the art for the presently disclosed embodiments.

The current state of the art fails to test individuals for predispositions to tooth decay and instead, health care providers issue general warnings and information in a one size fits all approach. There is a long felt need in the art for improved means of testing individuals to provide appropriate intervention to at risk individuals.

Dental caries (also known as tooth decay) remains the most common chronic disease of childhood and adulthood. In children, dental caries are five times more common than asthma and seven times more common than environmental allergies, with more than 40% of children exhibiting caries when they enter kindergarten. In 2005, it was estimated that dental health care costs were approximately $84 billion, of which 60% or about $50 billion were related to treatment of dental caries. Although overall caries prevalence has declined over the last 40 years, dental caries in the primary dentition and mean caries rates in children ages 2-11 has increased markedly over the past 12 years. Childhood caries is a severe public health issue because of associated health problems and because disparities in oral health have led to substantially higher average disease prevalence among children in poverty and in under-served racial and ethnic groups. These issues are of such concern that in 2005, the American Academy of Pediatrics made children's oral health one of their top areas of focus.

The etiology of dental caries has been studied for many years. Multiple factors contribute to a person's risk for caries, including: 1) environmental factors such as diet, oral hygiene, fluoride exposure and the level of colonization of cariogenic bacteria and 2) host factors such as salivary flow, salivary buffering capacity, position of teeth relative to each other, surface characteristics of tooth enamel and depth of occlusal fissures on posterior teeth. In spite of all that is known about this disease, there are still individuals who appear to be more susceptible to caries and those who are extremely resistant, regardless of the environmental risk factors to which they are exposed, implying that genetic factors also play an important role in caries etiology. This conclusion is supported by studies in both humans and animals, with the most compelling evidence coming from studies of twins reared apart in which investigators found significant resemblance within monozygotic (MZ) but not dizygotic (DZ) twin pairs for percentage of teeth and surfaces restored or carious and estimated the genetic contribution to caries as 40%. Other recent studies of twins reared together estimated the heritability for caries, adjusted for age and gender, as ranging from 45-64%.

### BRIEF SUMMARY OF THE INVENTION

The present invention overcomes shortfalls in the related art by presenting an unobvious and unique combination, configuration and use of molecular tests for testing individuals for genetic predispositions for tooth decay or caries.

While various prior art molecular tests do review aspects of genes TAS2R38 and TAS1R2, the following SNPs: rs635808, rs17057381, rs4251631, rs118500320, rs9793739, rs1383934, rs11099896, and any other SNP and/or gene linked to caries predisposition, the present invention overcomes shortfalls in the art by sequencing various genes, such as TAS2R38 and TAS1R2, the following SNPs: rs635808, rs17057381, rs4251631, rs118500320, rs9793739, rs1383934, rs11099896, and any other SNP and/or gene linked to caries predisposition, as part of a molecular test to identify those individuals having a genetic risk for caries.

In general, a molecular test for genetic components of caries development in primary and permanent dentition identifies individuals predisposed to caries. In one disclosed embodiment, DNA from the patient's saliva is isolated and sequences in those regions where single nucleotides polymorphisms have a predicted “high risk” role for caries development. The relevant pools of genes are sequenced together, with semiconductor-sequencing technology on a microchip, and a report of genetic risk is generated upon the alleles detected in the hot regions. The assessment of caries risk from DNA isolated from saliva enhances the implementation of targeted strategies, and thereby contribute to early dental caries prevention.

These and other aspects of the present invention will become apparent upon reading the following detailed description in conjunction with the associated drawings.

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

All the above references and U.S. patents and applications are incorporated herein by reference. Aspects of the invention can be modified, if necessary, to employ the systems, functions and concepts of the various patents and applications described above to provide yet further embodiments of the invention.

These and other changes can be made to the invention in light of the above detailed description. In general, the terms used in the following claims, should not be construed to limit the invention to the specific embodiments disclosed in the specification, unless the above detailed description explicitly defines such terms. Accordingly, the actual scope of the invention encompasses the disclosed embodiments and all equivalent ways of practicing or implementing the invention under the claims.

While certain aspects of the invention are presented below in certain claim forms, the inventors contemplate the various aspects of the invention in any number of claim forms.

In general, TAS2R38 is a bitter receptor gene which is commonly associated with PROP (6-n-propylthiouracil) sensitivity, super tasting and alcoholism. TAS1R2 is another taste receptor gene, but is known as a sweet taste receptor. Thus, the use of these two genes in an unobvious departure from traditional wisdom, especially considering the opposite bitter-sweet relationship between TAS2R38 and TAS1R2. Taste receptor TAS1R2 is known as a Type 1 sweet receptor and taste receptor TAS1R2 is known as a Type 2 bitter receptor.

In one aspect of the invention, a molecular test for TAS2R38 and/or TAS1R2, the following SNPs: rs635808, rs17057381, rs4251631, rs118500320, rs9793739, rs1383934, rs11099896, and any other SNP and/or gene linked to caries predisposition, is administered to a biological sample from a patient. If found, a caries intervention system is implemented.

Contemplated embodiments comprise sequencing or testing for a gene or SNP from the group comprising: TAS2R38, TAS1R2, the following SNPs: rs635808, rs17057381, rs4251631, rs118500320, rs9793739, rs1383934, rs11099896 or any other SNP and/or gene linked to caries predisposition.

The term “non-risk individuals” include those individuals who show SNPs genotypes—not linked—to caries predisposition genes. Contemplated embodiments disclose the following SNPs as linked to caries predisposition: rs635808, rs17057381, rs4251631, rs118500320, rs9793739, rs1383934, and rs11099896.

Items

Disclosed embodiments include the following items:

Item 1. A method for testing a predisposition for caries, the method comprising the steps of:
a) obtaining a sample of biological material from a patient;
b) obtaining a sequence of TAS2R38 and TAS1R2, the following SNPs: rs635808, rs17057381, rs4251631, rs118500320, rs9793739, rs1383934, rs11099896, and any other SNP and/or gene linked to caries predisposition, from the biological material from the patient;
c) comparing the obtained sequence of TAS2R38 and TAS1R2, the following SNPs: rs635808, rs17057381, rs4251631, rs118500320, rs9793739, rs1383934, rs11099896, and any other SNP and/or gene linked to caries predisposition to that of a normal individual; and
d) wherein the patient's obtained sequence of TAS2R38 and TAS1R2, the following SNPs: rs635808, rs17057381, rs4251631, rs118500320, rs9793739, rs1383934, rs11099896, and any other SNP and/or gene linked to caries predisposition is different than that of a non-risk individual indicates a predisposition to caries.

Item 2. The method of item 1 wherein the biological sample is taken from the saliva of a patient and the saliva is placed upon a semiconductor sequencing microchip and the semiconductor sequencing microchip is attached to a special purpose computer to obtain the sequence of TAS2R38 and TAS1R2, the following SNPs: rs635808, rs17057381, rs4251631, rs118500320, rs9793739, rs1383934, rs11099896, and any other SNP and/or gene linked to caries predisposition.

Item 3. The method of item 1 wherein an intervention of caries prevention is administered to a patient having an indicated predisposition to caries.

Item 4. A method of testing a genetic predisposition for caries, the method comprising the steps of:

a) obtaining a sample of saliva from a patient;
b) sequencing the sample of saliva to obtain sequencing of alleles mapped to TAS2R38 and TAS1R2;
c) wherein observed alleles mapped to TAS2R38 indicates a predisposition to caries;
d) wherein observed alleles mapped to TAS1R2 indicates a predisposition to caries; and
e) wherein observed alleles mapped to the following SNPs: rs635808, rs17057381, rs4251631, rs118500320, rs9793739, rs1383934, rs11099896, and any other SNP and/or gene linked to caries predisposition, indicates a predisposition to caries.

Item 5. The method of item 5 wherein the sample of saliva is placed upon a semiconductor sequencing microchip and the semiconductor sequencing microchip is processed with a special purpose computer to obtain the sequencing of alleles mapped to TAS2R38 and TAS1R2, the following SNPs: rs635808, rs17057381, rs4251631, rs118500320, rs9793739, rs1383934, rs11099896, and any other SNP and/or gene linked to caries predisposition.

Item 6. A method of testing a genetic predisposition for caries, the method comprising the steps of:

a) obtaining a sample of saliva from a patient;
b) testing the saliva to confirm the presence of TAS2R38 and TAS1R2; the following SNPs: rs635808, rs17057381, rs4251631, rs118500320, rs9793739, rs1383934, rs11099896, and any other SNP and/or gene linked to caries predisposition;
c) wherein the presence of TAS2R38 indicates a genetic predisposition to caries;
d) wherein the presence of TAS1R2 indicates a genetic predisposition to caries; and
e) wherein observed alleles mapped to the following SNPs: rs635808, rs17057381, rs4251631, rs118500320, rs9793739, rs1383934, rs11099896, and any other SNP and/or gene linked to caries predisposition, indicates a predisposition to caries.

Item 7. The method of item 6 wherein the saliva is tested by use of a semiconductor sequencing microchip and the semiconductor sequencing microchip is processed with a special purpose computer.

Item 8. The method of item 6 wherein the testing includes a search for alleles of TAS2R38 and TAS1R2 and the following SNPs: rs635808, rs17057381, rs4251631, rs118500320, rs9793739, rs1383934, rs11099896, and any other SNP and/or gene linked to caries predisposition.

What is claimed is:

1. A method for testing a predisposition for caries, the method comprising the steps of:
a) obtaining a sample of biological material from a patient;
b) obtaining a sequence of TAS2R38, TAS1R2, and the following single-nucleotide polymorphisms (SNPs): rs635808, rs17057381, rs4251631, rs118500320, rs9793739, rs1383934, and rs11099896 from the biological material;
c) comparing the obtained sequence of TAS2R38 and TAS1R2, and the following SNPs: rs635808, rs17057381, rs4251631, rs118500320, rs9793739, rs1383934, rs11099896; to that of a non-risk individual; and
d) wherein the patient's obtained sequence of TAS2R38 and TAS1R2 and the following SNPs: rs635808, rs17057381, rs4251631, rs118500320, rs9793739, rs1383934, and rs11099896 is different than that of a non-risk individual indicates a predisposition to caries.

2. The method of claim 1 wherein the biological sample is taken from the saliva of a patient and the saliva is placed upon a semiconductor sequencing microchip and the semiconductor sequencing microchip is attached to a special purpose computer to obtain the sequence of TAS2R38 and TAS1R2 and the following SNPs: rs635808, rs17057381, rs4251631, rs118500320, rs9793739, rs1383934 and rs11099896.

3. The method of claim 1 wherein an intervention of caries prevention is administered to a patient having an indicated predisposition to caries.

4. A method of testing a genetic predisposition for caries, the method comprising the steps of:
a) obtaining a sample of saliva from a patient;
b) sequencing the sample of saliva to obtain sequencing of alleles mapped to TAS2R38 and TAS1R2 and the following SNPs: rs635808, rs17057381, rs4251631, rs118500320, rs9793739, rs1383934 and rs11099896;
c) wherein observed alleles mapped to TAS2R38 indicates a predisposition to caries;
d) wherein observed alleles mapped to TAS1R2 indicates a predisposition to caries: and
e) wherein observed alleles mapped to one or more of the following SNPs: rs635808, rs17057381, rs4251631, rs118500320, rs9793739, rs1383934 or rs11099896, indicates a predisposition to caries.

5. The method of claim 4 wherein a the sample of saliva is placed upon a semiconductor sequencing microchip and the semiconductor sequencing microchip is processed with a special purpose computer to obtain the sequencing of alleles mapped to TAS2R38 and TAS1R2 and the following SNPs: rs635808, rs17057381, rs4251631, rs118500320, rs9793739, rs1383934 and rs11099896.

6. A method of testing a genetic predisposition for caries, the method comprising the steps of:
a) obtaining a sample of saliva from a patient;
b) testing the saliva to confirm the presence of TAS2R38, TAS1R2, or one of the following SNPs: rs635808, rs17057381, rs4251631, rs118500320, rs9793739, rs1383934 or rs11099896;
c) wherein the presence of TAS2R38 indicates a genetic predisposition to caries;
d) wherein the presence of TAS1R2 indicates a genetic predisposition to caries; and
e) wherein observed alleles mapped to one of the following SNPs: rs635808, rs17057381, rs4251631, rs118500320, rs9793739, rs1383934, rs11099896, indicates a predisposition to caries.

7. The method of claim 6 wherein the saliva is tested by use of a semiconductor sequencing microchip and the semiconductor sequencing microchip is processed with a special purpose computer.

8. The method of claim 6 wherein the testing includes a search for alleles of TAS2R38 or TAS1R2, or the following SNPs: rs635808, rs17057381, rs4251631, rs118500320, rs9793739, rs1383934, or rs11099896.

9. The method of claim 8 wherein the testing includes a search for any SNP and/or gene linked to caries predisposition.
